(19) United States
(12) Patent Application Publication
(10) Pub. No.: US 2003/0212127 A1
(43) Pub. Date: Nov. 13, 2003

(54) Method of treating actinic keratosis

(75) Inventors: Bradley P. Glassman, Fairfield, NJ (US); Dileep Bhagwat, Bronxville, NY (US); Daniel Glassman, Fairfield, NJ (US)

Correspondence Address: COVINGTON & BURLING, ATTN: PATENT DOCKETING, 1201 PENNSYLVANIA AVENUE, N.W., WASHINGTON, DC, 20004-2401, US

(73) Assignee: Bradley Pharmaceuticals, Inc.

(21) Appl. No.: 10/143,680

(22) Filed: May 9, 2002

(51) Int. Cl. A61K 31/355; A61K 31/17; A61N 1/30

(52) U.S. Cl. 514/458; 514/588; 604/20

(57) ABSTRACT

Described is a novel approach for treating actinic keratosis which involves the use of urea in a dermatological composition. The urea composition can be included in pre-treatment, treatment and post-treatment steps. Also described are novel topical compositions for the treatment step containing a combination of urea and a pharmaceutical agent for treating actinic keratosis, such as a caustic agent, 5-fluorouracil or a photosensitizing agent.

### FIELD OF THE INVENTION

The present invention relates to an improved method of treating actinic keratosis. This invention changes the focus of treating actinic keratosis from a singular event to a comprehensive procedure, which includes the use of a composition containing urea as a principal component and can include pre-treatment, treatment and post-treatment. Treatment may involve concurrent or non-concurrent topical applications.

### BACKGROUND OF THE INVENTION

Actinic Keratoses (AKs) are very common, precancerous lesions that arise on photodamaged skin. Extensive sun exposure and skin type are the most important factors in their development.

The term actinic means that development of the lesions results from exposure to ultraviolet (UV) radiation, the primary source of which is sunlight. Keratosis is a general term for skin lesions characterized by overgrowth and thickening of the stratum corneum. There is a strong correlation between sun exposure and the occurrence of AKs. Commonly affected sites are the balding scalp, forehead, face, ears, neck, and back of the forearms and hands. Although most AKs develop on the head and upper extremities, they can also occur on the legs or anywhere where there has been excessive exposure to UV radiation. Also, the lesions can develop as a result of UV light exposure from artificial sources, such as tanning booths. Medical radiation exposure or exposure through occupational means may also cause AKs. After several years, a small percentage of AKs degenerate to squamous cell carcinomas. Thus, the lesions require careful evaluation and effective treatment.

Current treatments primarily include cryosurgery and the administration of topical fluorouracil (5-FU), as well as photodynamic therapy with photosensitizing agents such as, aminolevulinic acid hydrochloride (ALA) or hematoporphyrin. Cryosurgery refers to the freezing off certain sections of skin with, for example, liquid nitrogen. 5-FU, when applied topically, can help slow certain cells' division and growth. Photodynamic therapy involves the administration of a photosensitizing agent to a subject, including administration of a precursor of a photosensitizing agent such as ALA, and subsequent irradiation with light of the target cells or tissue of the subject. The photosensitizing agent preferentially accumulates in the target cells, namely cells or tissues that are more rapidly proliferating or growing than other cells or tissues in the target environment. Other treatments include chemical peeling, dermabrasion, curettage and desiccation, and laser surgery.

Although the current treatments are somewhat effective, they can severely damage adjacent, healthy skin due to the fact that, in part, it is very difficult to determine diseased skin from healthy skin tissue. As such, it is possible for either current AK treatments to either miss an affected, pre-cancerous area as well as to harm healthy skin, adjacent to the AK spot. In addition, the active ingredients of some currently used therapies often do not penetrate or reach all the areas they need to treat due to the fact that AK's are associated with very tough, hardened areas of skin.

There is a need for products and methods to improve the outcome of AK treatment by enhancing the penetration of active ingredient in the desired areas, while ensuring that adjacent, healthy skin tissue is preserved.

### SUMMARY OF THE INVENTION

The present invention provides for an improved treatment for actinic keratosis, by using a combination of a topical urea composition, typically containing about 10 to about 60 wt-%, about 21 to about 40 wt-%, or about 40 wt % urea in conjunction with each of the current therapies now available. The use of urea compositions can be incorporated into current AK therapy encompassing three phases of treatment (pre-treatment, treatment, post-treatment).

Accordingly, the present invention provides a method for treating actinic keratosis on an area of skin of a human patient which includes the following: (a) pre-treating the area of skin by applying to the area a composition containing from about 10 to about 60 wt-% of urea and the balance being dermatologically acceptable excipients; and (b) administering treatment for the actinic keratosis concurrently with a urea composition. The method can further include the use of a composition containing about 10 to about 60 wt-% of urea during post-treatment. When used during treatment, urea composition can be separate from or include the treatment for the actinic keratosis. The urea compositions can further include antioxidants.

In one embodiment, treatment for the actinic keratosis can include cryosurgery, removal or excision with a scalpel, dermabrasion, laser surgery, electrosurgical skin resurfacing, irradiation, and administration of a therapeutically effective amount of a pharmaceutical agent, or a combination thereof. The pharmaceutical agent can include a caustic agent, a photosensitizing agent, 5-fluorouracil, masoprocol, retinoids, α-hydroxy acids, interferon, or a combination thereof.

The present invention also includes novel topical compositions containing about 10 to about 60 wt-% of urea, a therapeutically effective amount of a pharmaceutical agent for treatment of actinic keratosis, and the balance being dermatologically acceptable excipients. In one embodiment, the pharmaceutical agent includes a caustic agent, a photosensitizing agent, 5-fluorouracil, masoprocol, retinoids, α-hydroxy acids, interferon, or a combination thereof. The novel compositions can further include antioxidants.

### DETAILED DESCRIPTION OF THE INVENTION

The present invention relates to changing the treatment of actinic keratosis from a singular event to pre-treatment, treatment and optionally post-treatment of actinic keratosis, using a dermatological composition containing urea in amounts ranging from 10 to 60 wt-%, 21-40 wt-%, or about 40 wt-%. Treatment may involve concurrent or non-concurrent topical applications.

Urea

Pre-Treatment Phase

The invention provides methods using urea for the treatment of actinic keratosis, which includes a pre-treatment with about 10 to about 60 wt-%, about 21 to about 40 wt-%, or about 40 wt-% urea. Pretreatment with urea may assist an individual administering therapy in viewing an AK to be treated. For example, pre-treatment with urea, a keratolytic and softening agent, may expose hidden or non-visible AK by removing scaly, dry skin as well as softening tough skin. By allowing an individual administering therapy a better view of the AK to be treated, pretreatment with urea may allow therapy to be directed to an entire AK, as well as limit therapy directed at healthy surrounding tissue. In addition, the keratolytic and softening properties of urea may assist an individual administering treatment, e.g. a physician, to correctly differentiate between AK and “sun spots”, which may appear to be AK. Thus, pretreatment with urea can prevent treatment of “sun spots” and other healthy skin, which, if treated, would lead to the destruction of healthy skin. Therefore, urea pre-treatment provides the skin to be supple and clean in preparation for the treatment.

In addition, urea pre-treatment can provide increased moisture content of skin to be treated. Urea has been long recognized as a cosmetic ingredient in formulations acting as a humectant and moisturizer. Dermatological compositions containing from 21 to 40 wt-% urea for treating dry scaly skin have been described in U.S. Pat. No. 5,919,470, which is incorporated herein by reference. Additionally, there have been reports of keratolytic activity attributed to urea with the ability at high concentrations to solubilize and denature protein. A striking effect is upon the water-binding capacity of the horny layer of the skin: pieces of normal horny layer, or scales from ichthyotic or psoriatic skin that have been soaked in 30% urea solution take up much more water. In maintaining the flexibility of the horny layer and the softness of the skin, the water content of the horny layer matters much more than its oil content. As such, urea pretreatment can increase drug penetration.

The duration of pre-treatment with urea can vary over any period of time. In general, the duration of pre-treatment provides for adequate freeing of debris and softening and moisturizing of the skin. A healthcare professional should be able to readily determine an appropriate duration of urea pre-treatment. In one embodiment of the invention, urea can be used for up to four weeks prior to treatment. Longer durations of administration of urea pre-treatment can also be used.

Treatment Phase

In addition to pre-treatment, about 10 to about 60 wt-% urea, about 21 to about 40 wt-%, or about 40 wt-% urea may be used concurrently with treatment of AK. When treatment of AK includes a pharmaceutical agent, the pharmaceutical agent may be administered in the same composition as urea or may be administered in a composition separate from the urea. Regardless of whether the treatment for AK includes a pharmaceutical agent, all of currently used treatments for AK can be enhanced through the concurrent use of urea. For example, concurrent administration of urea, can enhance the skin's absorption of a pharmaceutical agent for treatment of AK, as well as potentiate the therapeutic effect of the agent. By way of example, concurrent administration of urea can enhance penetration of 5-FU into skin cells, as well as increase the uptake of a photosensitizing agent. For other treatments, such as chemical peeling, cryosurgery, curettage and desiccation, dermabrasion, and laser surgery, concurrent treatment with urea can continue to keep the skin free of debris, allowing an individual administering treatment to minimize adverse affect on health skin, as well as maximize treatment of an AK. The use of concurrent urea treatment can enhance effectiveness of treatment with a pharmaceutical because the tissue will take up more of the pharmaceutical agent, and the pharmaceutical agent or other treatment will be directed to the right areas.

The duration of treatment will vary depending on the nature and type of treatment. For example, the duration of cryosurgery can take only seconds, while the duration of a therapy including a photosensitizing agent can take days from the initial administration of the agent to irradiation with UV light.

Post-Treatment

In addition to pre-treatment and concurrent treatment, about 10 to about 60 wt-%, about 21 to about 40 wt-%, or about 40 wt-% urea may be used as post-treatment for AK. Urea can keep the skin free from debris, allowing an individual, e.g. a health care professional, to observe potential regrowth of the AK, and thus determine whether additional treatment is indicated. Additionally, post-treatment with urea can protect open wounds created by treatment from infection. This is because of urea's antimicrobial activity. For example, high concentrations of urea, such as 40%, are also known to have an antibacterial effect. At these strengths the antibacterial effects are said to be similar to those of antibiotics, with the further advantage that all the common organisms are susceptible and the possibility of resistant strains need not be seriously considered. Dermatological compositions containing high amounts of urea have also been reported to have antifungal activity as described in co-pending application No. 10/103,213 of Mar. 20, 2002.

Post treatment of urea may continue as long as medically indicated. In one embodiment, post-treatment may include application of about 40 wt-% urea for up to 14 days.

The urea pre-treatment and concurrent treatment, or pre-treatment, concurrent treatment, and post-treatment can be combined with a wide variety of surgical and non-surgical therapies for actinic keratoses. The treatment method selected will depend on variables including medical status of the patient; lesion characteristics such as size, location, duration, and change in growth pattern; previous treatment; and certain anatomic locations such as the scalp and ear. Therapies, both surgical and non-surgical, may be combined in the treatment of actinic keratoses, as indicated by, for example, the type, extent, and location of the lesion.

Cryosurgery

Cryosurgery can be used according to a method of the invention to treat AK. Cryosurgery is a procedure utilizing cryogenic agents. Freezing temperatures of a cryogenic agent applied directly or indirectly to the skin can cause local destruction of tissue. Multiple or repeated treatments may be applied as indicated. Cryogens useful for cryosurgery include liquid nitrogen, which is the most often used cryogen for cryosurgery treatment of AK; other cryogens include, for example, solidified carbon dioxide; nitrous oxide; freons; and helium. The cryogens can be applied by a variety of known means including cotton-tipped applicator, open-spray, open-cone (confined spray), closed-cone, cryoprobe, and metal applicator. Typically a single freeze/thaw cycle is used for treating AK lesions, but more than one cycle may be used as indicated. Freeze time varies according to the cryogen used and the size of the AK lesion, but generally ranges from 3 to 60 seconds.

Other Forms of Surgery

Any surgical therapy useful for treatment of AK can be used with the invention. Surgical therapy useful for treatment of AK includes shave removal or excision with a scalpel, optionally followed by electrocautery to stop bleeding; dermabrasion, which can involve sanding off the top layers of the lesion with a rapidly rotating brush; laser surgery with, for example a carbon dioxide laser, to remove the skin to the desired depth; and electrosurgical skin resurfacing, using radiofrequency energy to remove skin layers without heat.

5-Fluorouracil

5-fluorouracil (5-FU) can be used according to a method of the invention to treat AK. 5-FU can be applied as needed to effectively treat AK. Typically, effective treatment includes twice a day application for several weeks. Treatment should typically continue until the AK lesions “light up” and becomes red and crusted. The duration of treatment will vary according to the location and nature of the AK lesion. For example, facial AK lesions may require three to four weeks of treatment, while treatment of lesions on the arms may require six to eight weeks. In some cases, treatment may be continued for 12 weeks or longer. Topical 5-FU treatment can be repeated on several occasions as indicated.

5-FU is available as brand names Fluoroplex®, Carac®, and Efudex®, as either a cream or topical solution. A composition containing any concentration of 5-FU can be used. However, the concentration should be sufficient to treat AK while being tolerable to the subject. For example, 5-FU creams can contain about 0.5% to about 5% 5-FU or about 1% to about 5% 5-FU. Similar concentrations of 5-FU are also useful in topical solutions.

Photosensitizing Agents

One or more photosensitizing agents can be used according to a method of the invention to treat AK. Photosensitizing agents are photoactivatable compounds and complexes that, upon irradiation, cause damage to cells. It is believed that a photochemical reaction occurs when a photosensitizing agent is irradiated at a photoactivating wavelength. The photochemical reaction is believed to generate chemical disruptive species, such as an oxygen radical, that interacts with cell parts, such as cellular and nuclear membranes. Any photosensitizing agent that damages cells leading to cell death can be used with the present invention regardless of its mechanism or site of effect on the cell.

Photosensitizing agents are known, as are their respective photoactivation wavelengths. Radiation of the appropriate wavelength for a given photosensitizing agent may be administered by a variety of methods known to one skilled in the art. For example the radiation may include laser, nonlaser, or broad band radiation. Any device which generates the appropriate wave form may be used. For example, fiber optic instruments, arthroscopic instruments, or instruments which provide transillumination, as is known to one of ordinary skill in the art may be used.

Examples of classes of photosensitizing agents include, but are not limited to, hematoporphyrins, uroporphyrins, phthalocyanines, purpurins, acridine dyes, bacteriochlorophylls, bacteriochlorins, psoralen-based compounds and derivatives thereof. Specific photosensitizing agents include porfimer sodium (Photofrin®); synthetic diporphyrins and dichlorins; hydroporphyrins such as chlorins and bacteriochlorins of the tetra(hydroxyphenyl) porphyrin series; phthalocyanines (PC), with or without metal substituents, e.g., chloroaluminum phthalocyanine (CASP), with or without varying substituents; O-substituted tetraphenyl porphyrins (picket fence porphyrins); 3,1-meso tetrakis (o-propionamido phenyl) porphyrin; Verdins; Purpurins, tin and zinc derivatives of octaethylpurpurin (NT2),etiopurpurin (ET2), Chlorins, chlorin e6, mono-1-aspartyl derivative of chlorin e6, di-1-aspartyl derivative of chlorin e6, benzoporphyrin derivatives (BPD), benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin; sulfonated aluminum PC, disulfonated (AIPCS₂), tetrasulfonated derivative, sulfonated aluminum naphthalocyanines, naphthalocyanines, with or without metal substituents, with or without varying substituents, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, hematoporphyrin (HP), other naturally occurring porphyrins, 5-aminolevulinic acid and other endogenous metabolic precursors, benzonaphthoporphyrazines, cationic imminium salts, tetracyclines, 8-MOP (methoxsalen, xanthotoxin), 5-methoxypsoralen (5-MOP, bergaptin), 7-methylpyridopsoralen, isopsoralen, other isomeric and chemical derivative forms of psoralen, bis(di-isobutyloctadecylsiloxy)-2,3-naphthalocyanato silicon (isoBO--SiNc), and tin etiopurpurin (SnET2).

Photosensitizing agents useful for the invention include precursors of photoactivatable compounds or complexes. An example of a precursor of a photoactivatable compound useful for the invention is 5-aminolevulinic acid (5-ALA). 5-ALA is metabolically converted to the photosensitizing agent, protoporphyrin IX, after administration to a subject.

Photosensitizing agents can be administered in dosages or concentrations as known in the art. For example, useful photosensitizing agents can be administered systemically or topically. In one embodiment of the invention, photosensitizing agents are topically administered. In topical applications, the photosensitizing agent may be administered as a free compound or complex, or may be administered in various formulations, such as solutions, lotions, or creams as known in the art.

Some photosensitizing agents that can be useful for the invention are typically administered systemically. For example, porfimer sodium is typically parentally injected at a dose of 2 mg per kg of subject body weight. Other photosensitizing agents, such as methoxsalen, are administered systemically or topically. Topical methoxsalen is available as a 1% lotion (Oxsoralen®).

In one embodiment, 5-ALA is topically administered as a photosensitizing agent. 5-ALA is available under the brand name Levulan® as a topical solution, 20%, containing the hydrochloride salt of 5-ALA. 5-ALA is metabolically converted after uptake by cells to protoporphyrin IX, which can be photoactivated by blue light at 6-10.9 J/cm². Photoactivation typically occurs 14 to 18 hours after application of 5-ALA, while blue light treatment lasts about 17 minutes.

Regardless of whether a photosensitizing agent is typically administered systemically, the agent may be administered topically according to the invention as urea pretreatment and concurrent treatment can improve the ability of the agent to penetrate the skin and be delivered to the cells targeted for treatment.

Other Pharmacological Therapies

Any pharmacological therapy useful for treatment of AK can be used with the invention. Examples of additional pharmacological therapies useful for the treatment of AK include retinoids (vitamin A derivatives). α-Hydroxy acids and intralesional interferon injections may also be useful to treat AK.

Any AK treating effective route of administration can be used. For example, retinoids can be administered topically or systemically to treat AK. Topical retinoids are typically in the form of creams and include Retin A® and Avita®.

Caustic agents, such as trichloroacetic acid, phenol, or other known caustic agents can be used to treat AK according to a method of the invention. Treatment with caustic agents cause the top layer of skin to slough off and trigger new skin growth.

Compositions

The invention provides novel topical compositions comprising about 10 to 60 wt-%, about 21 to 40 wt-%, and about 40 wt-% urea; a therapeutically effective amount of a pharmaceutical agent useful for treating AK; and the balance being dermatologically acceptable excipients. The compositions may include antioxidants.

The topical compositions of the invention can include one or more pharmaceutical agents useful for treating AK. Pharmaceutical agents useful for the compositions of the invention include those agents that are administered topically, as the urea of the compositions can increase the penetration of pharmaceutical agents into the skin as discussed above. In one embodiment, the pharmaceutical agents are selected form the group consisting of 5-FU, photosensitizing agents, retinoids, interferons, α-hydroxy acids, and caustic agents. The photosensitizing agent can be, for example, 5-ALA, methoxsalen, porfimer, or verteporfin. The caustic agent can be, for example, trichloroacetic acid or phenol.

Novel topical compositions containing both urea and 5-fluorouracil are provided. The amount of urea may vary in this embodiment anywhere from about 10 to 60 wt-%, about 21 to 40 wt-%, and at about 40 wt-%. The amount of 5-fluorouracil includes that employed as approved for the current treatment. For example, the amount of 5-fluorouracil may vary in this composition from about 0.25 to about 10 wt-%, or about 0.5 to about 5 wt-%, or about 0.5 to about 2 wt-%.

The invention also provides novel topical compositions containing both urea and 5-aminolevulinic acid or a salt thereof. The amount of urea may vary in this embodiment anywhere from about 10 to about 60 wt-%, about 21 to about 40 wt-%, and at about 40 wt-%. The amount of 5-aminolevulinic acid or a salt thereof, such as the hydrochloride salt, includes that employed as approved for the current treatment. For example, the amount of 5-aminolevulinic acid or a salt thereof may vary in this composition from about 10 to about 30 wt-%, about 15 to about 25 wt-%, or about 20 wt-%.

5-Aminolevulinic acid and salts thereof can be unstable. Accordingly, in one embodiment, the 5-aminolevulinic acid or salt thereof is stored in a separate container in a dry state and is mixed with a urea composition prior to administration. For ease of mixture, the urea composition can be a solution. The separate container in which the 5-aminolevulinic acid or salt thereof can be any container that prevents degradation of the 5-aminolevulinic acid or salt thereof. The container can be, for example, an amber bottle.

The invention further provides novel topical compositions containing both urea and methoxsalen. The amount of urea may vary in this embodiment anywhere from about 10 to about 60 wt-%, about 21 to about 40 wt-%, and at about 40 wt-%. The amount of methoxsalen includes that employed as approved for the current treatment. For example, the amount of methoxsalen may vary in this composition from about 0.1 to about 2 wt-%, about 0.5 to about 2 wt-%, or about 1 wt-%.

Methoxsalen is relatively insoluble in water. Accordingly, in one embodiment of the invention, methoxsalen is added to an oil phase during preparation of the composition.

The topical compositions of the invention may include one or more antioxidants. Antioxidants include, but are not limited to, tocopherols (vitamin E), tocopherol derivatives, tocotrienols, ascorbic acid (vitamin C), ascorbic acid derivatives, carotenoids, vitamin A or derivatives thereof, butylated hydroxytoluene, butylated hydroxyanisole, gallic esters, flavonoids such as, for example, quercetin or myricetin, selenium, grape seed extract, catechins such as, for example, epicatechin, epicatechingallate, epigallocatechin or epigallocatechingallate, sulfur-containing molecules such as, for example, glutathione, cysteine, lipoic acid, N-acetylcysteine, chelating agents such as, for example, ethylenediamine tetraacetic acid or other customary antioxidants. In one embodiment, antioxidants are present in a composition of the invention at about 0.1 to about 20 wt-%.

One antioxidant of interest is vitamin E. The term “vitamin E” includes tocopherol (vitamin E) and derivatives thereof such as, for example, α-, β-, γ-, δ-, ε-, ζ₁, ζ₂, and 72 -tocopherol, and α-tocopherol acid succinate. Vitamin E is known as an antioxidant and protective vitamin for phospholipids of the cell membrane. It maintains the permeability and stability of the cell membrane, Lucy. Annals N. Y. Academy of Science 203, p. 4 (1972). It further has been known that vitamin E has a membrane-sealing effect. In erythrocytes, the simplest cells of the human body, there has been found that vitamin E provides a protective effect for the cell membrane. As with all antioxidants, vitamin E protects cells, including, epidermal cells which are susceptible to a wide range of oxidating events. In one embodiment, vitamin E is present in a composition of the invention at about 2.5 wt %.

The topical compositions of the invention can be formulated into any medium acceptable for dermatological application. For example, the compositions can be formulated into solutions, creams, lotions, and the like. Dermatologically acceptable excipients useful for the production of such formulations are known.

Dermatologically acceptable excipients include those described in U.S. Pat. No. 5,919,470. The excipients particularly include skin protectants which include a combination of semi-solid and liquid petroleum fractions. The semi-solid skin protectant is contained in about 5.5 to about 20 wt-% and includes petrolatum or a synthetic or semi-synthetic hydrocarbon of the same nature as petrolatum. Mixtures of such ingredients can also be used. The preferred semi-solid material is petrolatum, commercially available from a wide variety of sources.

The liquid portion skin protectant is a liquid petrolatum and contained in the composition in about 10 to about 20 wt-%. This material can include any synthetic or semi-synthetic oleaginous liquid fraction. A preferred embodiment is mineral oil, which is a liquid mixture of hydrocarbons obtained from petroleum.

Another preferred ingredient encompassed in the composition of the present invention is propylene glycol which may be contained up to about 5 wt-% in the composition, preferably in the range of from about 1 to about 5 wt-%.

In addition to the above embodiments, the present composition also contains dermatologically acceptable excipients, such as for example emulsifiers and thickeners. Among these are for example a C₁₆ to C₁₈ straight or branched chain fatty alcohols or fatty acids or mixtures thereof. Preferably these include cetyl alcohol, stearyl alcohol, stearic acid, palmitic acid, or mixtures thereof. Fatty acids or fatty alcohols may be present in from about 0.25 to 2 wt-%.

Another ingredient useful in the composition of the present invention may be glyceryl stearate, which is a monoester of glycerine and stearic acid, or other suitable forms of glyceryl stearate for example glyceryl stearate SE, which is a commercially available self-emulsifying grade of glycerol stearate that contains some sodium and/or potassium stearate. Glyceryl stearate may be in the composition anywhere from about 1 to about 3 wt-%.

Xanthan gum is another ingredient which may be used in the present invention. Xanthan gum is a high molecular weight heteropolysaccharide gum produced by pure-culture fermentation of a carbohydrate with Xanthomonas campestris. The gum is also commercially available from various sources.

As part of the dermatologically acceptable excipients, the composition includes thickeners which provide a high viscosity cream designed to remain in place upon application to the skin. Preferred thickeners include a mixture of a carbomer and triethanolamine. The mixture is combined together and added to the composition in an amount totaling anywhere from about 0.05 to 5 wt-%. Triethanolamine is purchased as Trolamine NF from BASF. The carbomers come in various molecular weights and identified by numbers. These are otherwise known as Carbopol. A preferred embodiment of the present invention is Carbopol 940. The carbomer or Carbopols are resins which are known thickening agents. They are homopolymers of acrylic acid crosslinked with an allyl ether of pentaerythritol, an allyl ether of sucrose or an allyl ether of propylene. The carbomer is present in the composition as a thickener and also is used to suspend and stabilize the emulsion. Although Carbopol 940 is preferably used in the present invention, other analogs may also be used such as carbomer 910, 2984, 5984, 954, 980, 981, 941 and 934. Carbopol ETD 2001, 2020, and 2050 and Ultrez 20 are also commercially available and can be used since they are similar in chemistry and function.

Typical compositions employed in the present invention are for example:

| Ingredient | Approximate Wt- % |
|---|---|
| urea | 40 |
| 5-FU | 0.5-2 |
| petrolatum or a synthetic or semi-synthetic | 5.5-20 |
| hydrocarbon, or a semi-solid mixture thereof | |
| liquid petrolatum or synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| water QS | 100 |

| Ingredient | Approximate Wt- % |
|---|---|
| urea | 40 |
| 5-FU | 0.5-2 |
| Vitamin E | 2.5 |
| petrolatum or a synthetic or semi-synthetic | 5.5-20 |
| hydrocarbon, or a semi-solid mixture thereof | |
| liquid petrolatum or synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| water QS | 100 |

| Ingredient | Approximate Wt- % |
|---|---|
| urea | 30 |
| 5-FU | 0.5-2 |
| petrolatum or a synthetic or semi-synthetic | 5.5-20 |
| hydrocarbon, or a semi-solid mixture thereof | |
| liquid petrolatum or a synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| mixture of a carbomer and triethanolamine | 0.05-5 |
| water QS | 100 |

| Ingredient | Approximate Wt- % |
|---|---|
| urea | 30 |
| 5-FU | 0.5-2 |
| Vitamin E | 2.5 |
| petrolatum or a synthetic or semi-synthetic | 5.5-20 |
| hydrocarbon, or a semi-solid mixture thereof | |
| liquid petrolatum or a synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| mixture of a carbomer and triethanolamine | 0.05-5 |
| water QS | 100 |

| Ingredient | Approximate Wt- % |
|---|---|
| urea | 10-60 |
| methoxsalen | 0.1-2 |
| petrolatum or a synthetic or semi-synthetic | 5.5-20 |
| hydrocarbon, or a semi-solid mixture thereof | |
| liquid petrolatum or synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| water QS | 100 |

| Ingredient | Approximate Wt- % |
|---|---|
| urea | 10-60 |
| Methoxsalen | 0.1-2 |
| Vitamin E | 2.5 |
| petrolatum or a synthetic or semi-synthetic | 5.5-20 |
| hydrocarbon, or a semi-solid mixture thereof | |
| liquid petrolatum or synthetic or semi-synthetic | 10-20 |
| oleaginous liquid fraction, or a mixture thereof | |
| C₁₆₋₁₈ aliphatic straight or branched chain fatty | 0.25-2 |
| alcohol or fatty acid, or a mixture thereof | |
| propylene glycol | 1-5 |
| glyceryl stearate | 1-3 |
| xanthan gum | 0.01-0.5 |
| water QS | 100 |

An example of a composition according to the invention where aminolevulirnc acid is to be added to a urea solution prior to administration to a subject is as follows:

| Ingredient | To make 1 g of product |
|---|---|
| Vehicle | |
| Urea | 400.0 mg |
| Propylene Glycol | 50.0 mg |
| Buffer QS to pH 6-8 | |
| P. Water QS | 800.0 mg |
| Drug (Dry state) | |
| Aminolevulinic Acid | 200.0 mg |

Vitamin E can be added to the Vehicle. To adjust for weight percent of ingredients, the amount of vitamin E added is taken out of the water added.

A typical formulation representing a particular embodiment of the present invention is illustrated as follows:

| Ingredient | % W/W |
|---|---|
| 5-Fluorouracil (5-FU) | 0.5 |
| Urea USP | 40.4 |
| Carbopol 940 | 0.20 |
| Petrolatum | 8.94 |
| Mineral oil | 7.1 |
| Glyceryl stearate | 2.88 |
| Cetyl alcohol | 1.63 |
| Propylene glycol | 2.00 |
| Xanthan gum | 0.05 |
| Trolamine | 0.10 |
| Purified water Q.S. | 100.00. |

A typical formulation representing a particular embodiment of the present invention is illustrated as follows:

| Ingredient | % W/W |
|---|---|
| 5-Fluorouracil (5-FU) | 0.5 |
| Urea USP | 40.4 |
| Vitamin E | 2.5 |
| Carbopol 940 | 0.20 |
| Petrolatum | 8.94 |
| Mineral oil | 7.1 |
| Glyceryl stearate | 2.88 |
| Cetyl alcohol | 1.63 |
| Propylene glycol | 2.00 |
| Xanthan gum | 0.05 |
| Trolamine | 0.10 |
| Purified water Q.S. | 100.00. |

A typical formulation representing another particular embodiment of the present invention is illustrated as follows:

| Ingredient | % W/W |
|---|---|
| Methoxsalen | 1.0 |
| Urea USP | 40.4 |
| Carbopol 940 | 0.20 |
| Petrolatum | 8.94 |
| Mineral oil | 7.1 |
| Glyceryl stearate | 2.88 |
| Cetyl alcohol | 1.63 |
| Propylene glycol | 2.00 |
| Xanthan gum | 0.05 |
| Trolamine | 0.10 |
| Purified water Q.S. | 100.00. |

A typical formulation representing another particular embodiment of the present invention is illustrated as follows:

| Ingredient | % W/W |
|---|---|
| Methoxsalen | 1.0 |
| Urea USP | 40.4 |
| Vitamin E | 2.5 |
| Carbopol 940 | 0.20 |
| Petrolatum | 8.94 |
| Mineral oil | 7.1 |
| Glyceryl stearate | 2.88 |
| Cetyl alcohol | 1.63 |
| Propylene glycol | 2.00 |
| Xanthan gum | 0.05 |
| Trolamine | 0.10 |
| Purified water Q.S. | 100.00. |

We claim:

1. A method for treating actinic keratosis on an area of skin of a patient comprising:
(a) pre-treating the area by applying a composition comprising from about 10 to about 60 wt-% of urea, and the balance being dermatologically acceptable excipients;
(b) administering a treatment for actinic keratosis concurrently with the composition comprising from about 10 to about 60 wt-% urea..

2. The method of claim 1, wherein the urea composition contains an antioxidant.

3. The method of claim 2, wherein the antioxidant is vitamin E.

4. The method of claim 1, where further post-treating the area by applying a composition comprising from about 10 to about 60 wt-% of urea and the balance being dermatologically acceptable excipients.

5. The method of claim 4, wherein the urea composition comprises an antioxidant.

6. The method of claim 5, wherein the antioxidant is vitamin E.

7. The method of claim 1, wherein the treatment for actinic keratosis is selected from the group consisting of cryosurgery, removal or excision with a scalpel, dermabrasion, laser surgery, electrosurgical skin resurfacing, irradiation, administration of a therapeutically effective amount of a pharmaceutical agent, and a combination thereof.

8. The method of claim 7, wherein the pharmaceutical agent is selected from the group consisting of a caustic agent, a photosensitizing agent, 5-fluorouracil, masoprocol, retinoids, α-hydroxy acids, interferon, and a combination thereof.

9. The method of claim 7, wherein the treatment comprises administering a photosensitizing pharmaceutical agent and subsequent irradiation of the area.

10. The method of claim 9, wherein the photosensitizing agent is 5-aminolevulinic acid or a salt thereof.

11. The method of claim 9, wherein the photosensitizing agent is methoxsalen or a derivative thereof.

12. The method of claim 8, wherein the pharmaceutical agent is 5-fluorouracil.

13. The method of claim 7, wherein the pharmaceutical agent is included in a composition comprising from about 10 to about 60 wt-% of urea and the balance being dermatologically acceptable excipients.

14. The method of claim 13, wherein the pharmaceutical agent is included in a composition comprising from about 21 to about 40 wt-% of urea and the balance being dermatologically acceptable excipients.

15. The method of claim 14, wherein the pharmaceutical agent is included in a composition comprising about 40 wt-% of urea and the balance being dermatologically acceptable excipients.

16. The method of claim 1, wherein the pre-treatment urea composition and the concurrent urea composition comprise from about 21 wt-% to about 40 wt-% urea.

17. The method of claim 1, wherein the pre-treatment urea composition and the concurrent urea composition comprise about 40 wt-% urea.

18. A topical composition comprising:
(a) about 10 to about 60 wt-% urea;
(b) a therapeutically effective amount of a pharmaceutical agent for treatment of actinic keraotosis; and
the balance being dermatologically acceptable excipients.

19. The composition of claim 14, further comprising an antioxidant.

20. The composition of claim 19, wherein the antioxidant is present in the composition at about 0.1 to about 20 wt-%

21. The composition of claim 19, wherein the antioxidant is vitamin E.

22. The composition of claim 21, wherein the vitamin E is present in the composition at about 2.5%.

23. The composition of claim 18, wherein the pharmaceutical agent is selected from the group consisting of a caustic agent, a photosensitizing agent, 5-fluorouracil, masoprocol, retinoids, α-hydroxy acids, interferon, and a combination thereof.

24. The composition of claim 18, wherein the pharmaceutical agent is a photosensitizing agent.

25. The composition of claim 24, wherein the photosensitizing agent is aminolevulinic acid or a salt thereof.

26. The composition of claim 25, comprising about 15 to about 25 wt-% aminolevulinic acid or a salt thereof.

27. The composition of claim 25, wherein the composition is a solution.

28. The composition of claim 24, wherein the photosensitizing agent is methoxsalen.

29. The composition of claim 28, comprising about 0.1 to about 2 wt % methoxsalen.

30. The composition of claim 18, wherein the pharmaceutical agent is 5-fluorouracil.

31. The composition of claim 30, comprising about 0.5 to about 5 wt-% 5-fluorouracil.

32. The composition of claim 30, comprising about 0.5 to about 2 wt-% 5-fluorouracil.

33. The composition of claim 18, comprising about 21 to about 40 wt-% urea.

34. The composition of claim 18, comprising about 40 wt-% urea.

35. The composition of claim 18, wherein the excipients comprise skin protectants of an oleaginous nature derived from petroleum, emulsifiers and thickeners.

36. The composition of claim 35, wherein the skin protectants are a mixture of a semi-solid petrolatum or a synthetic or semi-synthetic hydrocarbon or a mixture thereof, and a liquid petrolatum or a synthetic or semi-synthetic oleaginous liquid derivative thereof, or a mixture thereof.

37. The composition of claim 36, wherein the semi-solid petrolatum is present in an amount from about 5.5. to about 20 wt-%.

38. The composition of claim 18, which composition further comprises up to 5 wt-% of propylene glycol.

39. The composition of claim 18, which composition further comprises a mixture of a carbomer and triethanolamine in a total amount from about 0.05 to about 5 wt-%.

40. A composition comprising:
(a) about 10 to about 40 wt-% urea;
(b) a therapeutically effective amount of a pharmacological agent for treatment of actinic keratosis;
(c) about 5.5 to about 20 wt-% petrolatum or a synthetic or semi-synthetic hydrocarbon, or a semi-solid mixture thereof;
(d) about 10 to about 20 wt-% of a liquid petrolatum or a synthetic or semi-synthetic oleaginous liquid fraction, or a mixture thereof;
(e) about 0.25 to about 2 wt-% of a C₁₆₋₁₈ aliphatic straight or branched chain fatty alcohol or fatty acid, or a mixture thereof;
(f) about 1 to about 5 wt-% propylene glycol;
(g) about 1 to about 3 wt-% glyceryl stearate;
(h) about 0.01 to about 0.5 wt-% xanthan gum;
(i) about 0.05 to about 5 wt-% of a mixture of a carbomer and triethanolamine; and
(j) the balance being water.

41. The composition of claim 40, comprising about 40 wt-% urea.

42. The composition of claim 40, further comprising an antioxidant.

43. The composition of claim 42, wherein the antioxidant is vitamin E.

44. The composition of claim 40, wherein the pharmaceutical agent is selected from the group consisting of a caustic agent, a photosensitizing agent, 5-fluorouracil, masoprocol, retinoids, α-hydroxy acids, interferon, and a combination thereof.

45. The composition of claim 40, wherein the pharmaceutical agent is a photosensitizing agent.

46. The composition of claim 45, wherein the photosensitizing agent is aminolevulinic hydrochloride or methoxsalen.

47. The composition of claim 40, wherein the pharmaceutical agent is 5-fluorouracil.
